SEQUENCE LISTING (3 sequences)

SEQ ID NO: 1 - DNA, 21 bp, Artificial Sequence
  Synthetic Forward primer for amplification of RCC1
aagaaggtgaaggtctcacac

SEQ ID NO: 2 - DNA, 20 bp, Artificial Sequence
  Synthetic Reverse primer for amplification of RCC1
gcacaacatcctccggaatg

SEQ ID NO: 3 - DNA, 21 bp, Artificial Sequence
  Synthetic RCC1 shRNA
tttctagtaggcaaagccagg

(19) United States
(12) Patent Application Publication
(10) Pub. No.: US 2016/0362686 A1
(43) Pub. Date: Dec. 15, 2016

(54) COMPOSITION INCLUDING RCC1-REGULATING SUBSTANCE FOR REDUCING SENESCENCE OF CELL OR SUBJECT AND METHOD OF REDUCING SENESCENCE OF CELL OR SUBJECT BY USING THE COMPOSITION

(71) Applicant: Samsung Electronics Co., Ltd., Suwon-si (KR)

(72) Inventors: Sungjin Ryu, Yongin-si (KR); Byungju Kim, Suwon-si (KR); Hyojei Choi, Seongnam-si (KR); Sangchul Park, Seongnam-si (KR)

(21) Appl. No.: 15/042,399

(22) Filed: Feb. 12, 2016

(30) Foreign Application Priority Data
Jun. 11, 2015 (KR) 10-2015-0082742

(51) Int. Cl. C12N 15/113

(52) U.S. Cl. CPC C12N 15/113; C12N 2320/30

(57) ABSTRACT

A method of controlling senescence in a cell by inhibiting regulator of chromosome condensation 1 (RCC1) gene expression or RCC1 protein activity in the cell.

### CROSS-REFERENCE TO RELATED APPLICATION

This application claims the benefit of Korean Patent Application No. 10-2015-0082742, filed on Jun. 11, 2015, in the Korean Intellectual Property Office, the disclosure of which is incorporated herein in its entirety by reference.

### BACKGROUND

1. Field

The present disclosure relates to a composition for reducing senescing levels in a cell or a subject and a method of reducing senescing levels in a cell or a subject by using the composition.

2. Description of the Related Art

Senescence or aging refers to a degenerative phenomenon associated with age-related declines. Regarding senescence in humans, deterioration of physiological activities may occur as people age while certain enzymatic activities or certain hormone secretion functions may increase. Cellular senescence is defined as a permanent halt in cell division. Senescence, also known as replicative senescence or cellular senescence, has been observed as an aging model at cellular level. When cells are successively cultured, the cells undergo a number of cycles of divisions, but they are no longer able to divide during cellular senescence. Senescent cells are actually resistant to programmed cell death, and some senescent cells have remained in a non-dividing state for years.

A nuclear transport system, which is associated with transport of substances between a nuclear and a cytoplasm of a cell, is required for maintaining cell functions. In the transport of cytoplasmic substances at a nuclear membrane boundary, modification of a nuclear pore complex (NPC), a nuclear transport receptor, a RanGTP gradient, and a transporting substance (e.g., i.e., cargo) may be involved. As cellular senescence progresses, functions of the nuclear transport system may also change. However, it is unknown whether such a change in the nuclear transport system according to cellular senescence is a cause or a result of cellular senescence. In addition, age-related factors of the nuclear transport system have not been identified.

Accordingly, a composition for reducing the senescence of a cell or a subject and a method using the composition are required, the composition including a substance that changes the nuclear transport system.

### SUMMARY

Provided is a composition for reducing senescing level in a cell or a subject, the composition comprising a substance that controls an expression of a chromosome condensation 1 (RCC1) gene or protein activity of the RCC1 gene.

Provided is a method of controlling a senescing level in a cell or a subject by inhibiting chromosome condensation 1 (RCC1) gene expression or RCC1 protein activity in the cell.

Additional aspects will be set forth in part in the description which follows and, in part, will be apparent from the description, or may be learned by practice of the presented exemplary embodiments.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1A displays cells stained with senescence-associated beta-galactosidase (SA β-gal).

FIG. 1B is a graph showing percentage (%) of the cells stained with SA β-gal.

FIG. 1C is a graph showing luminescence percentage (arbitrary unit/μg protein %) of the cells stained with SA β-gal.

FIG. 1D is an image of an immunoblotting analysis performed on cells that are treated with drugs;

FIG. 2A shows an image obtained by immunoprecipitating and then immunoblotting analysis performed on proteins of a model for senescent cells.

FIG. 2B is a graph showing relative amounts of RanGTP (%) calculated based on the image of FIG. 2A.

FIG. 3A is a graph showing quantitative PCR results analyzing amounts of mRNA of regulator of chromosome condensation 1 (RCC1) in a model for senescent cells.

FIG. 3B is an image of an immunoblotting analysis performed on proteins of the RCC1 in a model for senescent cells of human dermal fibroblasts (HDFs).

FIG. 3C is a graph showing a relative intensity of the proteins of the RCC1 with respect to an intensity of Actin based on results obtained by an immunoblotting analysis performed on proteins of the RCC1 in HDF, IMR-90, and HMEC that are subjected to successive cell division.

FIG. 4A is an image young cells that are transfected with shRNA of the RCC1 and stained with SA β-gal.

FIG. 4B is an image obtained by immunoblotting proteins of the young cells that are transfected with shRNA of the RCC1.

FIG. 5A is an image of senescent cells that are transfected with a RCC1-overexpressing virus and stained with SA β-gal.

FIG. 5B is a graph showing a relative number (%) of cells of transfected young and senescent cells.

FIG. 5C is an image obtained by immunoblotting proteins from transfected young and senescent cells.

FIG. 5D is an image of senescent cells that are transfected with the RCC1-V5 virus and stained with SA β-gal.

FIGS. 6A and 6B are graphs showing results of chromatin immunoprecipitation (ChIP) performed on young cells and senescent cells.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items.

An aspect of the present inventive concept provides a composition for controlling a senescing level in a cell or a subject, the composition including a material capable of controlling expression of regulator of chromosome condensation 1 (RCC1) genes or activity of RCC1 proteins.

The term “RCC1” may be used interchangeably with the term “Ran Guanine nucleotide Exchange Factor (RanGEF)”. The RCC1 may function as a guanine exchange factor of a Ras-related nuclear protein (Ran). The Ran may be involved in the nuclear transport of RNAs and proteins by using a nuclear pore complex. The RCC1 may bind to a guanosine diphosphate (GDP)-bound Ran (RanGDP) in a nuclear of a cell, to allow the exchange of intranuclear guanosine triphosphate (GTP) and GDP of the RanGDP, thereby generating a GTP-bound Ran (RanGTP). The control of the expression of the RCC1 or the activity of the RCC1 may also influence the control of the nuclear transport system.

The RCC1 gene may include an RCC1 protein-coding polynucleotide or a combination of a promoter and the RCC1 protein-coding polynucleotide. The RCC1 gene may have in humans, for example, a nucleic acid sequence of GenBank Accession No. NM_001048194. The RCC1 gene may have in mice, for example, a nucleic acid sequence of GenBank Accession No. NM_001197082. The promoter may include, for example, an RCC1 gene promoter, a cytomegalovirus (CMV) promoter, an SV40 promoter, an U6 promoter, a PGK promoter, and an elongation factor (EF)-1 promoter.

The term “expression of genes” refers to a process of synthesizing a gene product using gene information. The gene products may include a polypeptide, a protein, or RNA. Thus, the expression of the RCC1 genes refers to a process of synthesizing RCC1 proteins using the RCC1 genes. The expression of the RCC1 genes may be controlled by stimulating or inhibiting the expression of the RCC1 genes.

The RCC1 protein in humans may include, for example, an amino acid sequence of GenBank Accession No. NP_001041659. The RCC1 protein in mice may include, for example, an amino acid sequence of GenBank Accession No. NP_001184011. The RCC1 proteins may have activity of generating a RanGTP by exchanging GTP in a cell nucleus and GDP of a RanGDP. The activity of the RCC1 protein may be controlled by stimulating or inhibiting the activity of the RCC1.

A substance that controls the activity of the RCC1 protein may include a small-molecule compound, a nucleic acid, an ion, a protein, an antibody, or a combination thereof.

The substance that controls the expression of the RCC1 gene may include a substance that inhibits the expression of the RCC1 gene or a substance that overexpresses the RCC1 gene.

The substance that inhibits the expression of the RCC1 gene may include a small hairpin RNA (shRNA), a small interfering RNA (sRNA), a microRNA (miRNA), an antisense oligonucleotide, an inhibitor of a transcription factor of the RCC1 gene, or a combination thereof. The shRNA may be an RNA molecule having a hairpin structure and capable of inhibiting (or silencing) expression of a target gene through RNA interference. The sRNA may be an RNA molecule involved in RNA interference, and may inhibit gene expression the by inhibiting production of a specific protein. The shRNA or sRNA may have a length in a range of 10 nucleotides (hereinafter, referred to as ‘nt’) to 50 nt, 15 nt to 40 nt, 20 nt to 30 nt, or 21 nt to 23 nt. The shRNA may include a nucleic acid sequence of SEQ ID NO: 3 or a fragment thereof. The fragment be a polynucleotide having a nucleic acid sequence that includes a series of two or more nucleotides in the nucleic acid sequence of SEQ ID NO: 3. The fragment may have a length, for example, in a range of 2 nt to 50 nt, 3 nt to 40 nt, 4 nt to 30 nt, 5 nt to 20 nt, 6 nt to 15 nt, or 7 nt to 10 nt. The miRNA is a small-sized RNA that serves to control gene expression of an organism, and has a length in a range of about 17 nt to about 25 nt. The miRNA complementarily binds to an mRNA to increase or decrease expression of a particular protein. The antisense oligonucleotide is an ssDNA or an ssRNA complementary to a specific sequence, and complementarily binds to an mRNA to increase or decrease expression of a particular protein. The inhibitor of the transcription factor of the RCC1 gene may inhibit expression of the RCC1 gene. The substance that inhibits the expression of the RCC1 gene may, in the nucleus of the cell, increase an amount of the RanGDP and decrease an amount of the RanGTP. The amount of the RanGTP may be determined by any assay known in the art. The substance that inhibits the expression of the RCC1 gene may, in the nucleus of the cell, may decrease an amount of the RanGTP, so as to increase senescing levels in a cell or a subject.

The substance that overexpresses the RCC1 gene may include an expression vector including the RCC1 gene, a transcription factor of the RCC1 gene, or a combination thereof. The expression vector may be a virus vector, and the virus vector may include an adenoviral vector, a pSV vector, a pCMV vector, a vaccinia vector a lentiviral vector (e.g., pLKO.1 or pGIPZ), or a retroviral vector (e.g., pMSCV). The expression vector including the RCC1 gene may be configured to allow the expression of the RCC1 gene by including the RCC1 gene in the vector. The transcription factor of the RCC1 gene may be a specificity protein 1 (SP1) polypeptide. The SP1 polypeptide may serve as a transcription factor to stimulate the expression of the RCC1 gene. The SP1 may be encoded by, for example, a nucleic acid sequence of GenBank Accession No. NM_001251825 in humans or NM_013672 in mice. The SP1 may have, for example, an amino acid sequence of GenBank Accession No. NP_001238754 in humans or NP_038700 in mice.

The substance that overexpresses the RCC1 gene may, in the nucleus of the cell, decrease an amount of the RanGDP and increase an amount of the RanGTP. The increase in the amount of the RanGTP may lead to activation of the nuclear transport system. The substance that overexpresses the RCC1 gene may induce apoptosis of senescent cells, cell division of young cells, or a combination thereof.

The cell may be, for example, a nerve cell, an immune cell, an epithelial cell, a reproductive cell, a muscle cell, or a cancer cell. The cell may be a fibroblast or a cell of premature senescence. The cell of premature senescence may be a cell from a progeria patient.

The subject may include, for example, a human, cattle, a horse, a pig, a dog, a sheep, a goat, a rat, a mouse, a rabbit, or a cat.

The senescing levels in the cell or subject may be controlled by decreasing the senescing levels in the cell or subject. Senescence is a phenomenon that changes with the passage of time. Senescence in a cell or subject is indicated by changes in the cell or subject that may include, as compared with a reference cell or a reference subject, reduction in proliferation of a cell, accumulation of lipofuscin, increase in β-galactosidase activity, increase in mitochondrial reactive oxygen species, or a combination thereof. The reference cell may be a cell showing a doubling time (DT)≦1 day. The reference subject may be a subject containing at least a cell showing a doubling time (DT)≦1 day. The accumulation of lipofuscin may be determined by micrograph, as lipofuscin is granular yellow-brown pigment granules. The mitochondrial reactive oxygen species may be detected by any method known in the art, for example, fluorescent probes and immunoassays.

Senescence also may be indicated by any process that causes such changes in the cell or subject. The phenomenon of senescence in the cell or subject may include reduction in autophagy activity or reduction in mitochondrial membrane potential, or senescence may include any process that causes such a phenomenon.

A young cell or subject may include, as compared with the reference cell or the reference subject, increased proliferation of cells, decreased accumulation of lipofuscin, increased β-galactosidase activity, or a combination thereof. For example, A cell showing a DT≦1 day may be referred to as a young cell. A cell showing a DT≧14 days may be referred to as a senescent cell.

Controlling senescing levels in the cell or subject encompasses delaying or preventing senescence in a cell or subject, as well as converting a senescent cell or subject to young cell or subject (e.g., reverting a senescent cell or a subject having a senescent cell to a state that is similar with that of a pre-senescent cell or a subject having one or more pre-senescent cells). For example, the senescing level in the cell of individual may be reduced by increasing proliferation of a cell, decreasing β-galactosidase activity, decreasing accumulation of lipofucsin, reducing mitochondrial reactive oxygen species, or performing a combination thereof. The senescing levels in the cell or subject may be reduced by increasing autophagy activity, increasing mitochondrial membrane potential, or performing a combination thereof. The increase in proliferation of the cells may refer to reduction in cell division time or increase in the number of cell division. The β-galactosidase may be senescence-related β-galactosidase.

The composition may be used to prevent or treat a symptom or disease associated with senescence in the cell or subject. The symptom or disease associated with senescence in the cell or subject may include skin wrinkles, deterioration of wound regeneration, degenerative brain diseases (e.g., Alzheimer's disease, Parkinson's disease, and dementia), stroke, diabetes (e.g., type 2 diabetes), arthritis, atherosclerosis, heart diseases, hair loss, osteoporosis, sarcopenia, progeria, lysosome storage diseases, or a combination thereof. The symptom or disease associated with senescence in the cell or subject may include a disease associated with accumulation of lipofucsin. Lipofucsin is yellow-brown, autofluorescent pigment granules in cells, and may be used as an indicator of senescence. Lipofucsin is also referred to an aging pigment. The accumulation of lipofucsin may be shown in a patient with a retinal disease or in a liver, a kidney, or a heart of an aged person or a patient suffering from a wasting disease for a long period of time.

As used herein, the term “prevention” refers to inhibition or delay of onset of a symptom or disease associated with senescence by the administration of the composition. As used herein, the term “treatment” refers to the improvement or advantageous change of a symptom or disease associated with senescence by administering the composition.

The composition may be a pharmaceutical composition. The composition may further include a pharmaceutically acceptable carrier. The “pharmaceutically acceptable carrier” included in the composition may be a substance used in combination with an active ingredient to aid in application of the active ingredient, and for example, may be typically an inert substance. The pharmaceutically acceptable carrier may include an excipient, an additive, or a diluent, each of which is typically pharmaceutically acceptable. The pharmaceutically acceptable carrier may include, for example, at least one selected from a filler, a binder, a disintegrant, a buffer, a preservative, an antioxidant, a lubricant, a flavor, a thickener, a coloring agent, an emulsifier, a suspending agent, a stabilizer, and an isotonic agent.

The composition may include a “therapeutically effective dose” of the substance that controls the expression of the RCC1 gene or the activity of the RCC1 protein. The “therapeutically effective dose” in the composition refers to a dose sufficiently enough to exhibit a therapeutic effect when administered to a subject in need of treatment. The “therapeutically effective dose” may be determined by severity of a disease, a patient's age, weight, health condition, and gender, a patient's sensitivity to a drug, administration time, an administration route and a discharge rate, treatment duration, an element containing a drug used in combination with the composition of the present inventive concept or used at the same time as the composition of the present inventive concept, and other elements well known in the medical field. The “therapeutically effective dose” may include 0.01 mg to 10,000 mg, 0.1 mg to 1000 mg, 1 mg to 100 mg, 0.01 mg to 1000 mg, 0.01 mg to 100 mg, 0.01 mg to 10 mg, or 0.01 mg to 1 mg, per the composition.

A standard adult dose of the composition may be, for example, in a range of about 0.001 mg/kg to about 100 mg/kg, about 0.01 mg/kg to about 10 mg/kg, or about 0.1 mg/kg to about 1 mg/kg. The composition may be administered once a day, multiple times a day, or once a few days.

The composition may be may be orally administered, or may be parenterally administrated according to intravenous, intraperitoneal, subcutaneous, rectal, topical, or optical administration. Thus, the composition may be formulated in various forms, such as a tablet, a capsule, an aqueous solution, or a suspension. In the case of tablets for oral use, a lubricant, such as magnesium stearate, and one of lactose and an excipient, such as corn starch, may be included in the tablets. In the case of capsules for oral use, lactose and/or dried corn starch may be used as a diluent. When an aqueous suspension is necessary for oral use, an active ingredient may be combined with an emulsifier and/or a suspending agent. If necessary, a particular sweetener and/or a flavor may be included in the capsules. In terms of nerve, intramuscular, intraperitoneal, subcutaneous, and intravenous administration, a sterile solution containing an active ingredient may be generally prepared, and then, a pH of the sterile solution may be appropriately adjusted and buffered. In terms of intravenous administration, a total concentration of a solute may be adjusted to render a pharmaceutical preparation an isotonic property. The composition may be prepared in the form of an aqueous solution containing a pharmaceutically acceptable carrier, such as brine (pH of about 7.4). Such an aqueous solution may be used for local bolus injection to be administered to a muscle bloodstream or a nerve bloodstream of a patient.

The composition may further include at least one therapeutic agent other than the agents described above, to treat the diseases associated with senescence in the cell or subject.

Another aspect of the present inventive concept provides a method of controlling senescing levels in a cell or subject, the method including administering a substance to the cell or subject, wherein the substance controls expression of an RCC1 gene or activity of an RCC1 protein.

Descriptions of the RCC1, the RCC1 gene, the expression of the RCC1 gene, the RCC1 protein, the activity of the RCC1 protein, the substance that controls the expression of the RCC1 gene or the activity of the RCC1 gene, the cell, the subject, and senescence may be defined the same as those provided above.

The cell or subject may include cell or subject that suffer from a symptom or disease associated with senescence or have risks incident to a symptom or disease associated with senescence.

The administering of the substance may be performed by, for example, administering a standard adult dose in a range of about 0.001 mg/kg to about 100 mg/kg, about 0.01 mg/kg to about 10 mg/kg, or about 0.1 mg/kg to about 1 mg/kg. The administering of the substance may be performed once a day, multiple times a day, or once a few days during a period of one day to a period of one year. The administering of the substance may be performed according to methods known in the art. For example, the substance may be directly administered to a subject using any method, such as oral, intravenous, intramuscular, transdermal, mucous, intranasal, intratracheal, or subcutaneous administering method. The substance may be administered topically or systemically. The substance may be administered topically to a tissue including senescent cells.

The substance that controls the expression of the RCC1 gene may include a substance that inhibits the expression of the RCC1 gene or a substance that overexpresses the RCC1 gene. Descriptions of the substance that inhibits the expression of the RCC1 gene and the substance that overexpresses the RCC1 gene may be defined the same as those provided above. The substance that inhibits the expression of the RCC1 gene may increase senescing levels in the cell or subject by decreasing an amount of the RanGTP in the nucleus of the cell. The substance that overexpresses the RCC1 gene may increase an amount of the RanGTP in the nucleus of the cell, so as to induce apoptosis of senescent cells, cell division of young cells, or a combination thereof.

The method may be used to prevent or treat the symptom or disease associated with senescence in the cell or subject by controlling the senescing levels in the cell or subject.

The composition for controlling senescing levels in the cell or subject and the method of controlling senescing levels in the cell or subject by using the composition according to the exemplary embodiments above may efficiently control the senescing levels in the cell or subject, and accordingly, symptom or disease associated with senescence may be also efficiently prevented or treated.

Hereinafter, one or more embodiments will be described in more detail with reference to the following examples. However, these examples are for illustrative purposes only and are not intended to limit the scope of the one or more embodiments.

### EXAMPLE 1

### Control of Cellular Senescing Levels using a RCC1-Controlling Substance

1. Induction of Cellular Senescence by Inhibiting Nuclear Export

Cells were evaluated to determine whether cellular senescing levels depended on inhibition of nuclear import/export of substances between the nucleus and the cytoplasm of the cells.

First, human dermal fibroblast (HDF) M11 cells obtained from human neonatal foreskin were cultured in a medium supplemented with DEME, which contains glucose, glutamine, and pyruvate high concentrations, 10% (v/v) FBS, and 1× penicillin/streptomycin at a temperature of 37° C. in 5% CO₂, thereby obtaining young cells. Among the obtained young cells, cells having a passage number of 10 or less and a doubling time of about 1 day were selected.

The selected young cells were inoculated into a 6-well plate at a concentration of 20,000 cells/well. Then, wheat germ agglutinin (WGA) (Sigma, L0636) having a final concentration of 6 μg/ml, or Leptomycin B (LMB) (Sigma, L2913) having a final concentration of 0.1 μg/ml, were added to the inoculated cells. WGA is an inhibitor of nuclear import of substances, and LMB is an inhibitor of nuclear export of substances. Drugs, i.e., WGA and LMB, were treated on the medium every three days, and after 8 days of the treatment, the medium was replaced with a fresh medium that does not contain any drug. In addition, a negative control group was prepared in the same manner as described above, except that dimethyl sulfoxide (DMSO) (Sigma-Aldrich) was used, and a positive control group was prepared in the same manner as described above, except that cells that are not treated with a drug was successively to induce cellular senescence.

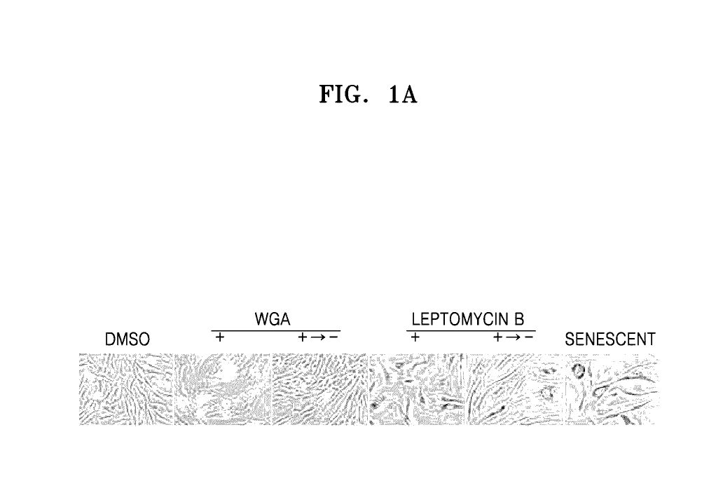

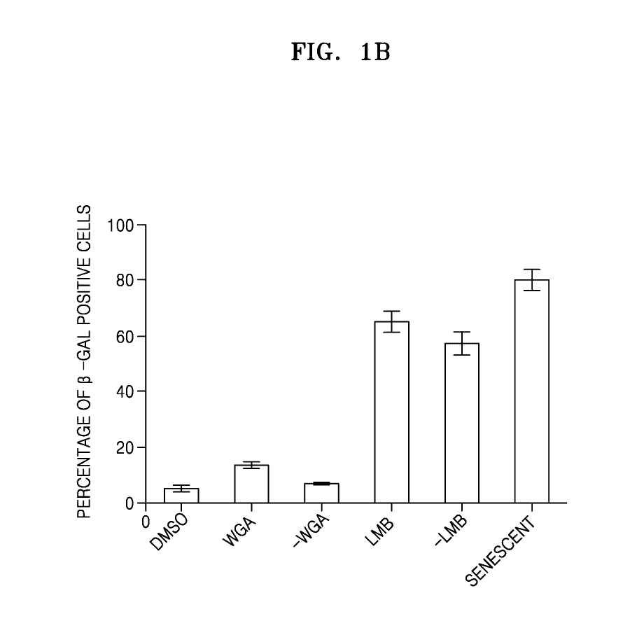

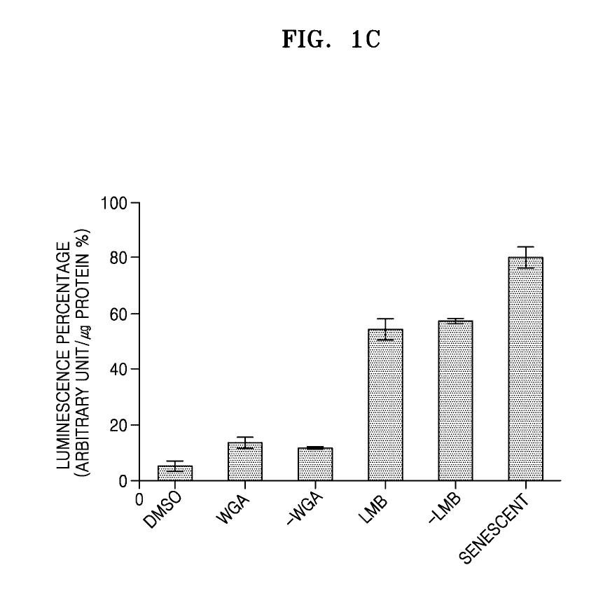

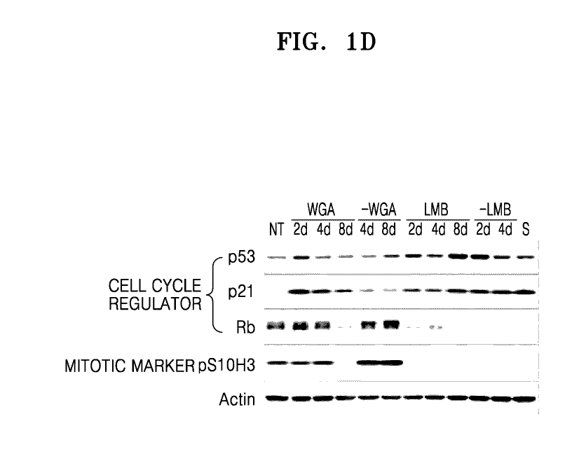

Senescent cells having increased activity of a senescence indicator, i.e., senescence-associated beta-galactosidase (SA β-gal), were stained with blue according to an X-gal staining process. To determine whether the administration of WGA or LMB influenced a decrease in senescing levels in the young cells, the cultured cells were stained using a SA β-gal staining kit (Cell Signaling Technology, Cat. No. 9860). Images obtained by observing stained cells with a microscope are shown in FIG. 1A (where “+” denotes drug administration, “+→−” denotes removal of drug after administration, and “senescent” denotes induction of cellular senescence by successive culture). In addition, percentage (%) of the stained cells and luminescence percentage (arbitrary unit/μg protein %) of the stained cells are shown in FIGS. 1B and 1C, respectively (where “−WGA” denotes removal of WGA after administration and “−LMB” denotes removal of LMB after administration). Accurate quantification of the SA β-gal based on the luminescence was obtained by correcting values, which was obtained using a luminometer (Promega GloMax®), to amounts of proteins according to a galacto-luminescence system. As shown in FIGS. 1B and 1C, it was confirmed that the percentage of the SA β-gal positive cells (i.e., the senescent cells) was increased in the LMB-treated group by about 60% compared with that of the SA β-gal positive cells in the negative control group and the WGA-treated group. The percentage of the SA β-gal positive cells in the −LMB group were maintained at a similar level with those in the senescent cell group.

In addition, to determine whether the drug treatment caused changes in amounts of protein markers for the cell division, anti-p53 antibodies (Santa Cruz, sc-126), anti-p21 antibodies (BD Pharmingen, #554228), anti-Rb antibodies (BD Pharmingen, #554136), anti-pS10H3 antibodies (Cell Signaling, #9701), and anti-Actin antibodies (Sigma, A1978) were used to carry out an immunoblotting analysis. An image consequently obtained by the immunoblotting process is shown in FIG. 1D (where “d” denotes days and “S” denotes induction of cellular senescence by successive culture). As shown in FIG. 1D, it was confirmed that the intensity of phosphorylated pS10H3 of Histone H3 was significantly decreased in the LMB group and the −LMB group and the intensity of p53 and p21, which are main proteins that inhibit the cell division, was significantly increased in the LMB group and the −LMB group.

Therefore, it was confirmed that cellular senescence was induced by inhibiting transportation of substances from the nucleus to the cytoplasm.

2. Confirmation of Amounts of RanGTP in Senescent Cells

To confirm RanGTP levels in the senescent cells, a model for senescence was prepared using replication-induced senescent cells, oncogene (ras)-induced senescent cells, and telomere dysfunction-induced senescent cells.

To prepare the replication-induced senescent cells, young cells (HFD cells with passage number of 9) prepared in the same manner as in Example 1.1 were successively cultured at a temperature of 37° C. in 5% CO₂, so as to prepare senescent cells (passage number of 52) having a doubling time of about 14 days. To prepare the oncogene (ras)-induced senescent cells, a pLenti CMV/TO RasV12 Puro (w119-1) vector (Addgene, #22262) in which a Ras V12 nucleic acid encoding an oncogenic Ras protein was cloned was introduced to the young cells. To prepare the telomere dysfunction-induced senescent cells, an expression vector (Pmscv TRF2 ΔBΔM obtained from Prof. Han, Jung-A of Kangwon National University) of a telomere repeat binding factor (TRF)2(ΔBΔM) (GenBank Accession No. NM_005652.4) was introduced to the young cells.

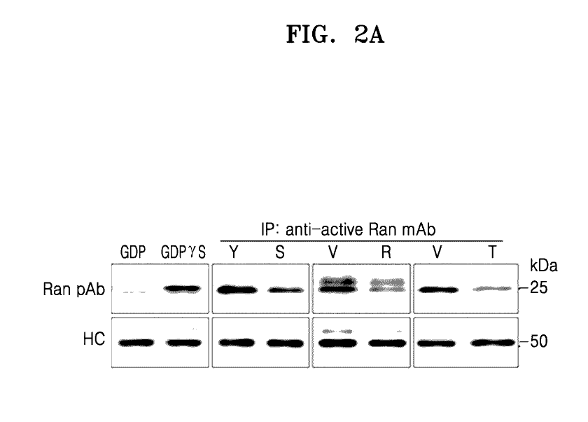

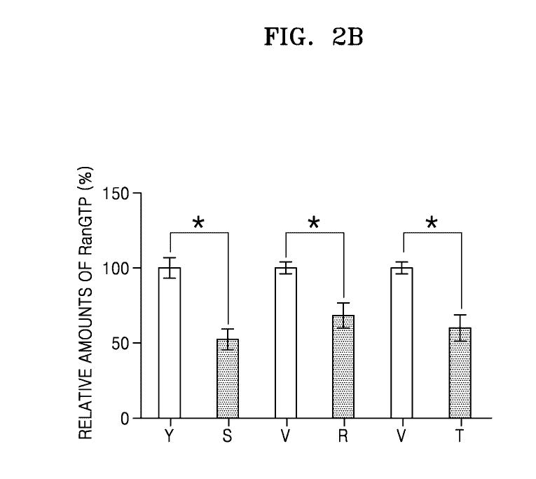

Proteins obtained from the prepared senescent cells were subjected to immunoprecipitate using anti-active Ran mouse antibodies (Abcam, ab173247), and proteins obtained from the immunoprecipitation were subjected to an immunoblotting analysis using anti-Ran rabbit polyclonal antibodies (Abcam, ab11693). A negative control group was prepared in the same manner as described above, except that a pLenti-puro vector (Addgene, #39481) was introduced. An image consequently obtained by the immunoblotting analysis is shown in FIG. 2A (where “Ran pAb” denotes an anti-Ran rabbit polyclonal antibody and “HC” denotes a heavy chain). Relative amounts (%) of the RanGTP calculated based on the image of FIG. 2A are shown in FIG. 2B (where “HDF” denotes human dermal fibroblasts, “Y” denotes young cells, “S” denotes senescent cells, “V” denotes cells including a vector introduced thereto, “R” denotes cells including oncogenic RasV12 introduced thereto, “T” denotes cells including TRF2(ΔBΔM) nucleic acids introduced thereto, and * denotes P<0.05).

As shown in FIGS. 2A and 2B, it was confirmed that the RanGTP levels in the replication-induced senescent cells, the oncogene-induced senescent cells, and the telomere dysfunction-induced senescent cells were reduced by about 30% to about 50% compared to those in the young cells. Thus, it was confirmed that the RanGTP levels were significantly reduced in the senescent cells compared to those in the young cells.

3. Confirmation of Reduction in Expression of RCC1 in a Model for Senescent Cells

The effect of senescence on amounts of the RCC1, which is an intracellular regulator that generates RanGTP, was analyzed in a model for each of the 3 types of the cells prepared in Example 1.2.

mRNA and proteins of the RCC1 were obtained from the model for senescent cells of HDF prepared in Example 1.2. In addition, IMR-90, which is a human lung-derived fibroblast, and a human mammary epithelial cell (HMEC) were used to induce cellular senescence according to the method described in Example 1.2. Then, mRNA of the RCC1 obtained from the model for senescent cells was subjected to a quantitative polymerase chain reaction (qPCR) by performing a primer set below.

(SEQ ID NO: 1)
Forward primer: 5′-AAGAAGGTGAAGGTCTCACAC-3′
(SEQ ID NO: 2)
Reverse primer: 5′-GCACAACATCCTCCGGAATG-3′

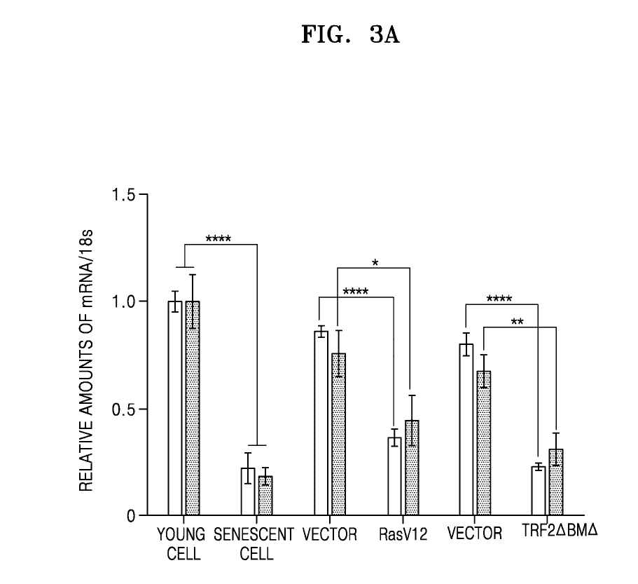

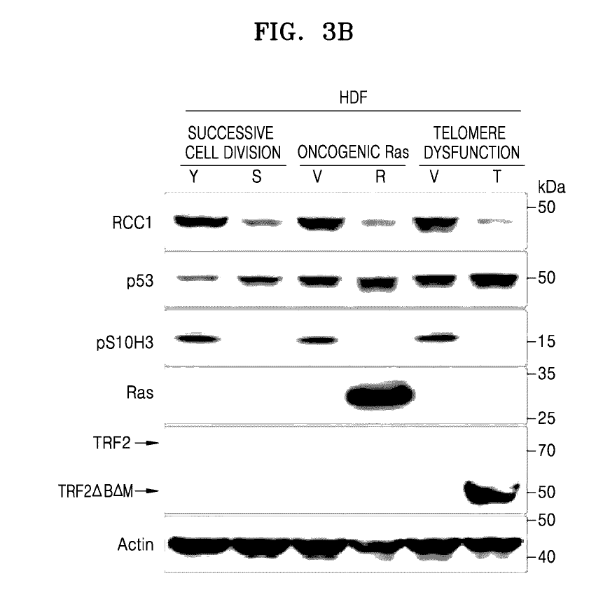

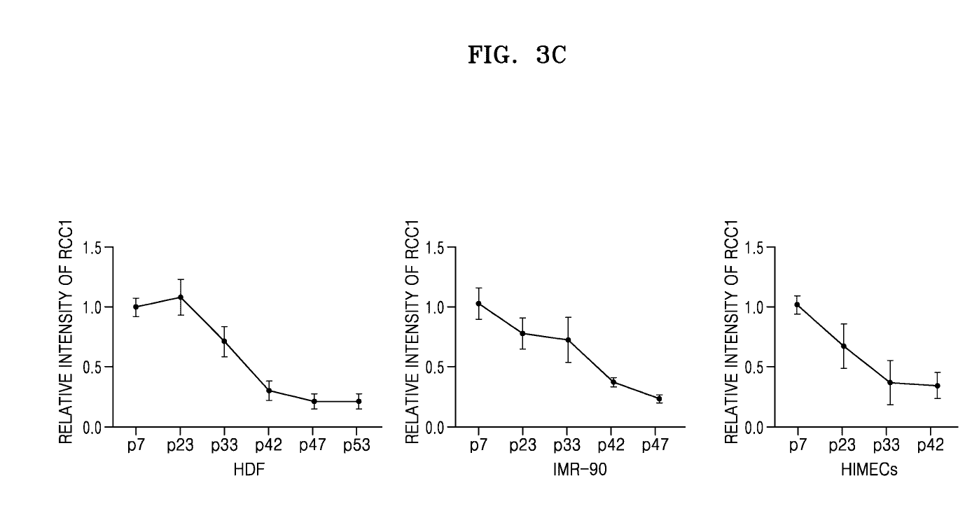

The results consequently obtained by the qPCR are shown in FIG. 3A (where □ denotes HDF cells, ▪ denotes IMR-90 cells, * denotes P<0.05, ** denotes P<0.01, *** denotes P<0.005, and **** denotes P<0.001). In addition, the anti-RCC1 antibodies (Epitomics #5134), anti-p53 antibodies (Santa Cruz, sc-126), anti-pS10H3 antibodies (Cell Signaling, #9701), anti-Ras antibodies (wBD Transduction Laboratories, #610001), anti-TRF2 antibodies (Santa Cruz, sc-52968) and anti-actin antibodies (Sigma, A1978) were used to perform an immunoblotting analysis. An image consequently obtained by the immunoblotting process is FIG. 3B (wherein “HDF” refers to human dermal skin trouble, “Y” indicates the young cells, “O” denotes senescent cells, “V” denotes cells including a vector introduced thereto, “R” denotes cells including oncogenic RasV12 introduced thereto, and “T” denotes cells including TRF2(ΔBΔM) nucleic acids introduced thereto). As shown in FIGS. 3A and 3B, amounts of mRNA and proteins of the RCC1 were both significantly reduced in the model for each of the 3 types of the cells.

HDF, IMR-90, and HMEC cells were induced to cellular senescence by successive cell division, and RCC1 proteins were subjected to an immunoblotting analysis using anti-RCC1 antibodies (Epitomics, #5134). Relative amounts (%) of the proteins of the RCC1 with respect to the intensity of actin according to a passage number are shown in FIG. 3C (where “p” denotes a passage number). As shown in FIG. 3C, when cellular senescence was induced by the successive cell division, it was confirmed the amount of the proteins of the RCC1 was decreased according to a passage number.

Thus, it was confirmed that the reduction of the RCC1 may be closely related to senescence.

4. Confirmation of Induction of Cellular Senescence According to Reduction in RCC1

To confirm whether cellular senescence can be induced by reducing the amount of the intracellular RCC1 and, accordingly, reducing the amount of the intracellular RanGTP, lentivirus pLKO.1 (Dharmacon, RHS3979-201872540 Seoulin BioScience) including an shRNA of the RCC1 having a nucleic acid sequence below was prepared.

(SEQ ID NO: 3)
RCC1 shRNA: 5′-TTTCTAGTAGGCAAAGCCAGG-3′

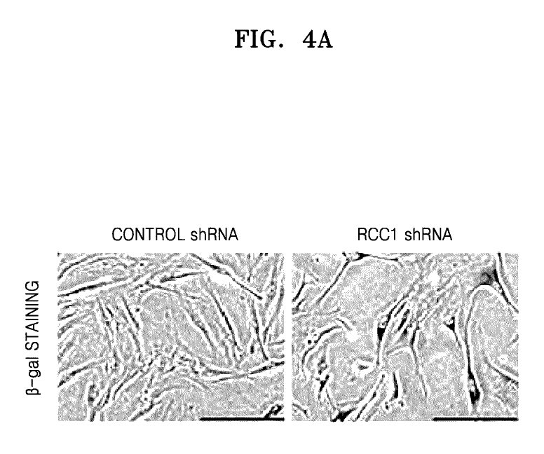

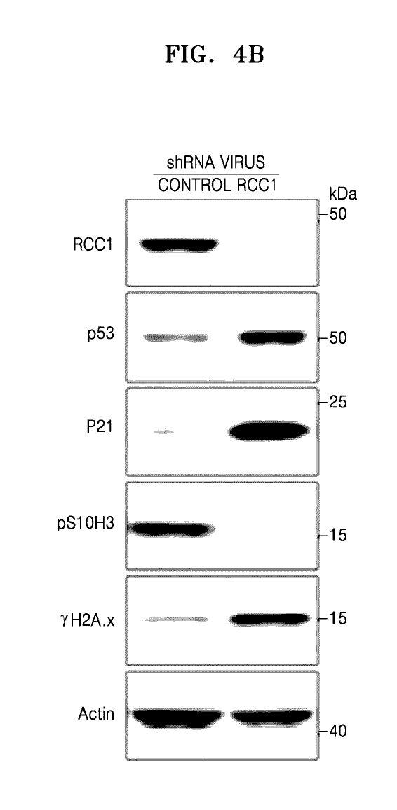

The young cells (i.e., HDFs) prepared in Example 1.1 were transfected with a negative control, such as shRNA (Dharmacon). The transfected cells were cultured for about 14 days at a temperature of 37° C. in 5% CO₂, and then, stained with the SA β-gal in the same manner as in Example 1.1, and images consequently obtained therefrom are shown in FIG. 4A. As shown in FIG. 4A, about 12.7% of SA β-gal positive cells were observed in a negative control group while about 53.4% of SA β-gal positive cells were observed in the cells transfected with the shRNA of the RCC1.

In addition, proteins were obtained from the cultured cells, and the proteins were subjected to an immunoblotting process using anti-p53 antibodies (Santa Cruz, sc-126), anti-p16 antibodies (Epitomics, #1963-1), anti-pS10H3 antibodies (Cell Signaling, #9701), anti-rH2A.x antibodies (Cell Signaling, #9718), and anti-RCC1 antibodies (Epitomics #5134). An image consequently obtained by the immunoblotting analysis is shown in FIG. 4B. As shown in FIG. 4B, it was confirmed that amounts of p53 and p16, which are proteins that inhibit the cell division, and rH2A.x, which is a DNA damage indicator, were increased in the cell group transfected with the shRNA of the RCC1 compared to the negative control group.

Therefore, it was confirmed that the inhibition of the RCC1 expression may cause cellular senescence in the young cells.

5. Confirmation of Effects of Overexpression of RCC1 in Senescent Cells

To confirm changes in senescent cells according to the overexpression of the RCC1 gene, a V5 virus (provided by Dr. Kalab Petr, NCI, NIH) including a nucleic acid (NM_001048194) that encodes the RCC1 gene was prepared.

The young HDF cells of Example 1.1 and the HDF cells of Example 1.2 having undergone successive cell division were prepared as young cells and senescent cells, respectively. Then, the prepared cells were transfected with the V5 virus. A negative control group was prepared in the same manner, except that a control virus (Addgene, #39481) was used.

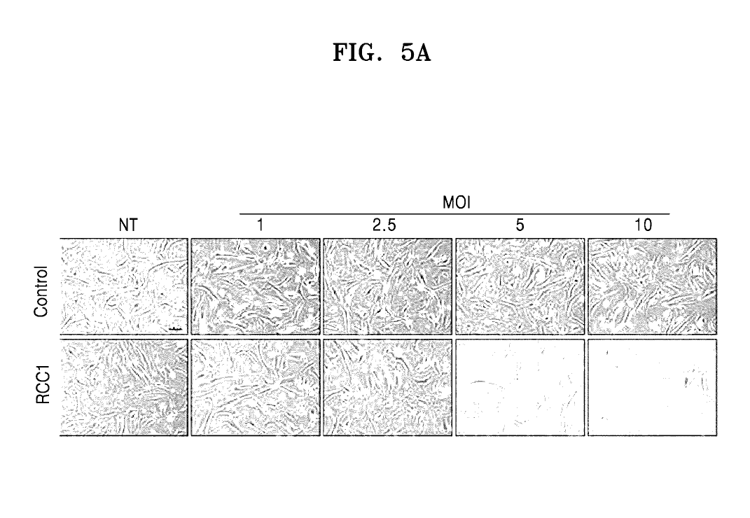

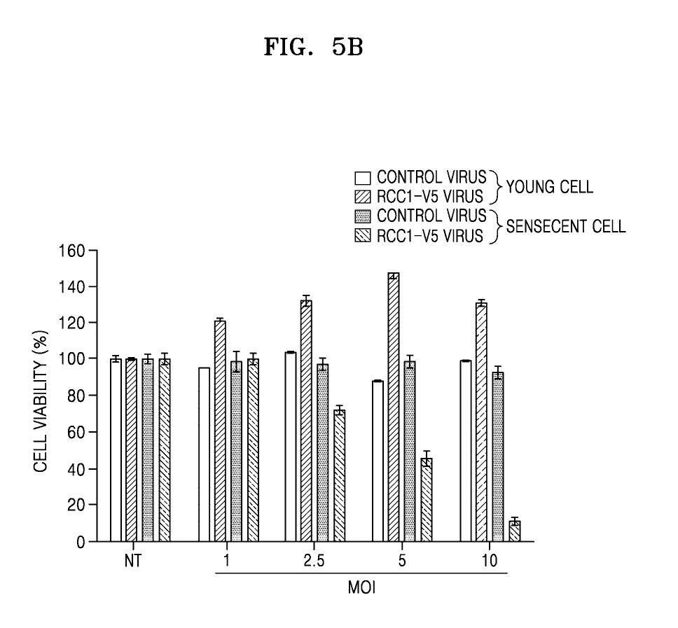

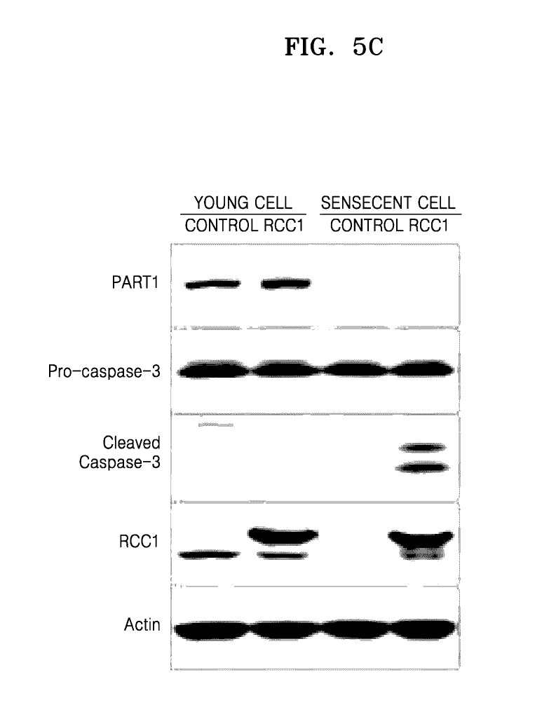

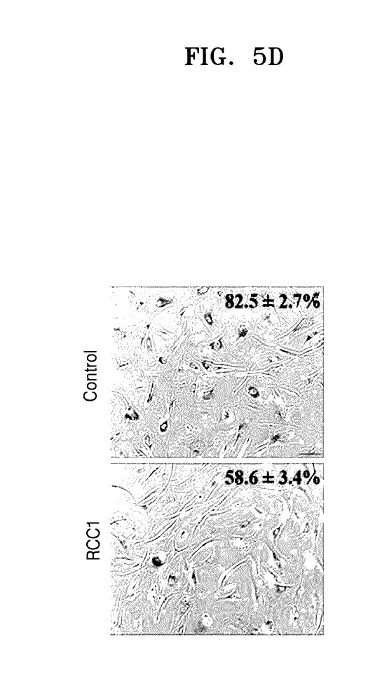

The transfected young and senescent cells were cultured for about 14 days at a temperature of 37° C. in 5% CO₂. Then, images of the transfected senescent cells according to the multiplicity of infection (MOI) are shown in FIG. 5A (where “NT” denotes cells not transfected with the virus, “control” denotes cells transfected with the control virus, and “RCC1” denotes cells transfected with the RCC1-overexpressing virus). In addition, cell viability (%) of the transfected young and senescent cells was calculated using MTT assay, and the results consequently obtained therefrom are shown in FIG. 5B. As shown in FIGS. 5A and 5B, apoptosis was induced in the senescent cells that overexpressed the RCC1 gene after 5 days of the transfection. Meanwhile, cell division was stimulated in the young cells that overexpressed the RCC1 gene.

Proteins were separated from the transfected young and senescent cells, and the separated proteins were subjected to an immunoblotting analysis using anti-PARP1 antibodies (Santa Cruz, sc-8007), anti-caspase-3 antibodies (Cell Signaling, #9665S), anti-RCC1 antibodies (Epitomics #5134), and anti-Actin antibodies (Sigma, A1978). An image consequently obtained by the immunoblotting analysis is shown in FIG. 5C. As shown in FIG. 5C, it was confirmed that the amount of the cleaved caspase-3, which is a representative marker of apoptosis, was increased in the senescent cells transfected with the RCC1-overexpressing virus and thus apoptosis was induced in the senescent cells.

In addition, the senescent cells were infected with respective RCC1-V5 virus and the control virus so that the MOI of the groups were 1. After about 4 weeks later, the infected senescent cells were stained with SA β-gal, and the results consequently obtained therefrom are shown in FIG. 5D (where “control” denotes cells transfected with the control virus and “RCC1” denotes cells transfected with the RCC1-V5 virus). As shown in FIG. 5D, it was confirmed that the amounts of SA β-gal positive cells were decreased by about 25%.

Therefore, it was confirmed that the overexpression of the RCC1 gene may lead to apoptosis specifically associated with the senescent cells, and the expression of the RCC1 gene may be controlled to decrease the senescing levels in the cell or subject. In addition, the control of the expression of the RCC1 gene may lead to prevention or treatment of diseases associated with senescence.

6. Confirmation of Transcription Factors that Control Expression of RCC1 in Senescent Cells

To confirm transcription factors that control the expression of RCC1 gene in the senescent cells, the HDF cells (passage number of 9) of Example 1.1 and the HDF cells (passage number of 52) of Example 1.2 undergone successive cell division were prepared as young cells and senescent cells, respectively.

The prepared cells were subjected to chromatin immunoprecipitation (ChIP) analysis by using anti-Sp1 antibodies (Millipore, #07-645).

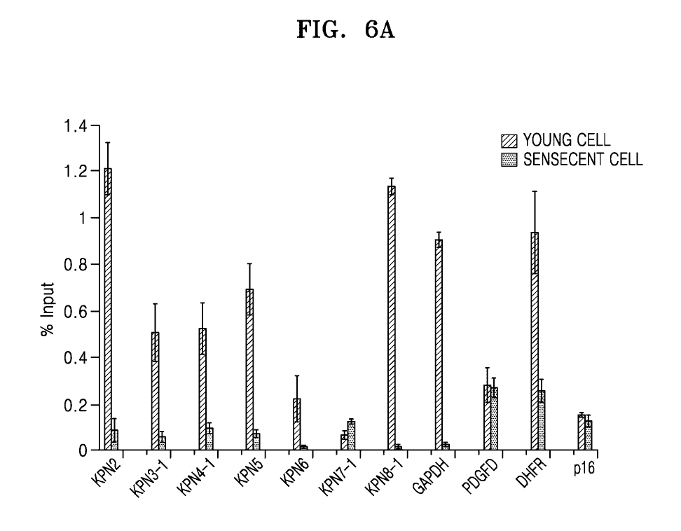

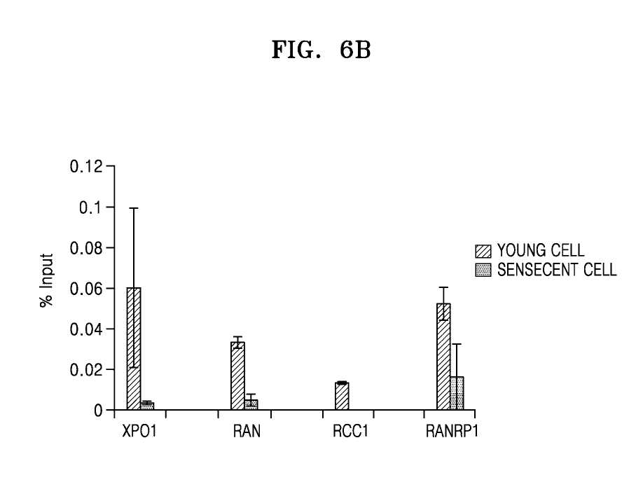

The results of the ChIP are shown in FIGS. 6A and 6B (percentage (%) input: percentage (%) based on the amount of chromatin used for the reaction). As shown in FIGS. 6A and 6B, several genes, in addition to the RCC1 gene, related to the nuclear transport system, were significantly less likely to bind to Sp1 in senescent cells than in young cells. Sp1 is a transcription factor in the senescent cells. Thus, it was confirmed that Sp1 can regulate the expression of the RCC1 in cells.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of controlling senescence in a cell, optionally in a subject, the method comprising inhibiting regulator of chromosome condensation 1 (RCC1) gene expression or RCC1 protein activity in the cell.

2. The method of claim 1, wherein inhibiting regulator of chromosome condensation 1 (RCC1) gene expression or RCC1 protein activity in the cell comprises administering a substance that controls RCC1 gene expression or RCC1 protein activity to the cell or to a subject comprising a cell.

3. The method of claim 2, wherein the cell or subject suffers from a symptom or disease associated with cell senescence.

4. The method of claim 2, wherein the substance that controls the expression of the RCC1 gene includes a substance that inhibits the expression of the RCC1 gene or a substance that causes overexpression of the RCC1 gene.

5. The method of claim 4, wherein the substance that inhibits expression of the RCC1 gene is a small hairpin RNA (shRNA), a small interfering RNA (sRNA), a microRNA (miRNA), an antisense oligonucleotide, an inhibitor of a transcription factor of the RCC1 gene, or a combination thereof.

6. The method of claim 4, wherein the substance that causes overexpression of the RCC1 gene comprises an expression vector including an RCC1 gene, a transcription factor of an RCC1 gene, or a combination thereof.

7. The method of claim 6, wherein the transcription factor of RCC1 genes is an SP1 polypeptide

8. The method of claim 4, wherein the substance that inhibits the expression of the RCC1 gene increases senescence in the cell by reducing an amount of a guanosine triphosphate (GTP)-bound Ras-related nuclear protein (Ran) (RanGTP) in the nucleus of the cell.

9. The method of claim 4, wherein the substance that causes overexpression of the RCC1 gene increases the amount of the RanGTP in the nucleus of the cell and induces apoptosis of senescent cells, cell division of young cells, or a combination thereof.

10. The method of claim 3, wherein the control of senescence in the cell is used to treat a symptom or disease associated with senescence of a cell.

11. The method of claim 1, wherein the control of senescence is a reduction of senescence.

12. The method of claim 11, wherein the reduction of senescing level in the cell or subject is an increase in cell proliferation, reduction in 3-galactosidase activity, a reduction in lipofuscin accumulation, a reduction in the amount of mitochondrial reactive oxygen species, or a combination thereof.

13. The method of claim 3, wherein the symptom or disease associated with cell senescence comprises skin wrinkles, deterioration of wound regeneration, degenerative brain diseases, stroke, diabetes, arthritis, atherosclerosis, heart diseases, hair loss, osteoporosis, sarcopenia, progeria, lysosome storage diseases, or a combination thereof.
